(19) United States
(12) Patent Application Publication
(10) Pub. No.: US 2009/0019957 A1
(43) Pub. Date: Jan. 22, 2009

(54) Method and system for detecting bombs in trash cans

(76) Inventors: Ishak Kalman Eisenman, Omer (IL); Arie Katua, Nes-ziona (IL)

Correspondence Address: KEVIN D. MCCARTHY;ROACH BROWN MCCARTHY & GRUBER, P.C., 424 MAIN STREET, 1920 LIBERTY BUILDING, BUFFALO, NY, 14202, US

(21) Appl. No.: 11/918,769

(22) PCT Filed: Apr. 20, 2006
(86) PCT No.: PCT/IL2006/000485

§ 371 Date: Oct. 18, 2007

(30) Foreign Application Priority Data
Apr. 21, 2005 (IL) 168181

(51) Int. Cl. B65F 1/14; G08B 21/02; G01N 9/02; G01N 27/00; G01N 33/22

(52) U.S. Cl. 73/866; 73/433; 340/540

(57) ABSTRACT

The present invention relates to a method and system for detecting bombs, which are thrown into a trash can. The system comprises: (a) at least one sensing unit for sensing predetermined characteristics of objects thrown into the trash can, and forming an indication signal when a characteristic at a level predefined to be suspected is sensed; and (b) a processing unit for receiving indication signals from said at least one sensing unit, evaluating said signals, and whenever determining emergency, forming an alert signal; characterized in that one of said sensing units is an RF detecting unit for detecting radio transmission from objects thrown into the trash can.

### FIELD OF THE INVENTION

The present invention relates to a method and system for detecting explosives that are placed in trash cans, particularly those located inside the airports and other public places.

### BACKGROUND OF THE INVENTION

Over the last few years, as a result of terrorist attacks, the issue of public security has gained major importance. Today, in all public places, there are many crucial target points, such as restrooms, flowerpots, dark corners, where terrorists may place explosives with no difficulty. Some of the above target points, such as restrooms or other public locations can be observed by security cameras and inspected by security personnel. However, observing and inspecting trash cans is very problematic, since trash cans have contents which are not easily visible. In airport terminals, in particular, there is a special team of security personnel called “sweepers”. This team is responsible for discovering explosives. However, even if a trash can is inspected and emptied many times a day, the problem still exists, since there is no control over what has been thrown into it.

A number of publications have tried to solve the problem of trash cans inspection and subsequent alert in cases when suspicious objects have been detected. However, all of the publications have failed to provide a satisfactory solution. For example, FR 2,744,705 presents a system of two-fold detection—weight and magnetic: the first, pneumatic detection, is provided by a pre-inflated air cushion equipped with a pressure transducer adjustable in the range from x kg to y kg, which is in turn connected to a microprocessor; the second consists of coils, that are attached to the interior wall of an envelope which set up an electromagnetic field. The magnetic indication is also provided to said microprocessor. Detection in each of the channels (weight as well as electromagnetic) involves triggering a sound alarm and a visual alarm with the option of communicating the alarm by means of a short-wave transmitter to a portable receiver. Although FR 2,744,705 may warn of possible danger, it still can not prevent the explosion. Furthermore, said publication does not teach means that are smart enough to distinguish between valid trash and typical explosives.

Another system for signaling the presence of a terrorist device is disclosed in BE 1009728. The proposed system is intended to warn people away from the proximity of a trash bin into which an abnormally heavy object has been thrown. The proposed system comprises a receptacle of a certain volume, equipped with lid at the top which permits the insertion of various waste items; an internal removable basket facilitating handling; an electro-pneumatic sensitive detector responding to the deposition of abnormal weight which would close a contact; a battery; a sounding alarm with minutes counter and a contact for communicating an alert notice to a control center. However, according to BE 1009728, the alarm is activated only in cases when an abnormally heavy object is deposited into the trash bin. This publication does not solve the problem of public safety in cases when terrorists use an explosive object of “normal” weight (i.e. within the range of normal objects usually thrown to the trash bin). BE 1009728 may detect a possible danger, but it still cannot prevent the explosion.

Therefore, there is a need to increase public safety, particularly in airport terminals and other public places, wherein many people congregate every day.

It is an object of the present invention to provide a system and method for detecting bombs and explosives placed in public trash cans, particularly within airport terminals and other public places.

It is another object of the present invention to provide a better automatic indication for detecting the placement of an explosive in a trash can.

It is sill another object of the present invention to provide a method and system which enable reducing the number of the security personnel allocated for inspecting trash cans.

It is a further object of the present invention to provide a method and system which is relatively inexpensive.

Other objects and advantages of the invention will become apparent as the description proceeds.

### SUMMARY OF THE INVENTION

The present invention relates to a method and system for detecting bombs and explosives placed in trash cans, located particularly inside the airports and other public places.

The system for detecting bombs which are thrown into a trash can, comprises: (a) at least one sensing unit for sensing predetermined characteristics of objects thrown into the trash can, and forming an indication signal when a characteristic at a level predefined to be suspected is sensed; and (b) a processing unit for receiving indication signals from said at least one sensing unit, evaluating said signals, and whenever determining emergency, forming an alert signal; characterized in that one of said sensing units is an RF detecting unit for detecting radio transmission from objects thrown into the trash can.

Preferably, the one or more sensing units are selected from a group comprising: (a) a weighing unit; (b) a metal detecting unit; (c) an E.M.F. (Electro Magnetic Field) detecting unit; and (d) an RF detecting unit.

Preferably, the system further comprises a frequency blocking unit for blocking at least one radio frequency or for blocking a predetermined range of radio frequencies.

Preferably, the system further comprises a transceiver for communicating with a control center, which is controlled by security personnel who are responsible for public safety.

Preferably, the system further comprises an alarm unit for initiating an audio or visual alarm whenever determining emergency.

Preferably, the system further comprises at least one battery or accumulator for providing electrical power to one or more units within said trash can.

Preferably, the trash can is further connected to a power supply providing electrical power to at least one unit within said trash can.

Preferably, the processing unit further sends one or more signals to one or more sensing units.

Preferably, the processing unit further sends one or more signals to the frequency blocking unit.

Preferably, the transceiver sends an alert signal to the control center when the change in weight, detected by means of the weighing unit is beyond a predetermined value or within a predetermined range.

Preferably, the transceiver sends an alert signal to the control center when one or more metal components in one or more objects thrown in the trash can are detected by means of the metal detecting unit.

Preferably, the transceiver sends an alert signal to the control center when the Electromagnetic Field inside the trash can is detected by means of the E.M.F. detecting unit.

Preferably, the transceiver sends an alert signal to the control center when the Radio Frequency transmission inside the trash can is detected by means of the RF detecting unit.

Preferably, the control center is further provided with means for stopping the audio or visual alarm.

Preferably, the audio or visual alarm is stopped at the trash can by means of at least one switch.

Preferably, the control center remotely adjusts and calibrates settings, parameters and sensitivity of the weighing unit, metal detecting unit, Electromagnetic Field detecting unit, RF detecting unit, Frequency blocking unit, alarm unit and transceiver.

Preferably, the settings, parameters and sensitivity of the weighing unit, metal detecting unit, Electromagnetic Field detecting unit, RF detecting unit, frequency blocking unit, alarm unit and transceiver are adjusted and calibrated at the trash can.

Preferably, the Frequency blocking unit comprises a metal shield for preventing any RF communication to or from the trash can, excluding communication of the transceiver.

Preferably, the weighing unit, metal detecting unit, Electromagnetic Field detecting unit, RF detecting unit, frequency blocking unit, alarm unit and the transceiver are in Sleep Mode, saving electrical power, until at least one object is thrown into the trash can.

Preferably, the processing unit in case of low power of at least one battery or accumulator sends an alert signal to the control center by means of the transceiver, warning the security personnel that said at least one battery or accumulator has to be replaced.

Preferably, the transceiver further comprises a programmable memory.

The method for detecting bombs which are thrown into a trash can, comprises: (a) sensing predetermined characteristics of objects thrown into the trash can by means of at least one sensing unit, and forming an indication signal by means of said at least one sensing unit when a characteristic at a level predefined to be suspected is sensed; and (b) receiving indication signals from said at least one sensing unit, evaluating said signals, and whenever determining emergency, forming an alert signal by means of a processing unit; characterized in that one of said sensing units is an REF detecting unit for detecting radio transmission from objects thrown into the trash can.

Preferably, the method further comprises providing a frequency blocking unit for blocking at least one radio frequency or for blocking a predetermined range of radio frequencies.

Preferably, the method further comprises providing a transceiver for communicating with a control center, which is controlled by security personnel who are responsible for public safety.

Preferably, the method further comprises providing an alarm unit for initiating an audio or visual alarm whenever determining emergency.

Preferably, the method further comprises providing at least one battery or accumulator for providing electrical power to one or more units within said trash can.

Preferably, the method further comprises connecting the trash can to a power supply, providing electrical power to at least one unit within said trash can.

Preferably, the method further comprises sending one or more signals to one or more sensing units by means of the processing unit.

Preferably, the method further comprises sending one or more signals to the frequency blocking unit by means of the processing unit.

Preferably, the method further comprises sending an alert signal to the control center by means of a transceiver, when the change in weight, detected by means of the weighing unit is beyond a predetermined value or within a predetermined range.

Preferably, the method further comprises sending an alert signal to the control center by means of a transceiver, when one or more metal components in one or more objects thrown in the trash can are detected by means of the metal detecting unit.

Preferably, the method further comprises sending an alert signal to the control center by means of a transceiver, when the Electromagnetic Field inside the trash can is detected by means of the E.M.F. detecting unit.

Preferably, the method further comprises sending an alert signal to the control center by means of a transceiver, when the Radio Frequency transmission inside the trash can is detected by means of the RF detecting unit.

Preferably, the method further comprises providing means for stopping the audio or visual alarm within the control center.

Preferably, the method further comprises stopping the audio or visual alarm at the trash can by means of at least one switch.

### BRIEF DESCRIPTION OF THE DRAWINGS

In the drawings:

FIG. 1A is a schematic block diagram of a conventional bomb;

FIG. 1B is a schematic illustration of a plurality of trash cans communicating with a control center, according to the present invention;

FIG. 1C is a schematic illustration of a trash can, according to an embodiment of the present invention;

FIG. 2 is a schematic block diagram of trash can units, according to an embodiment of the present invention;

FIG. 3A is a schematic illustration of trash can contents, according to an embodiment of the present invention;

FIG. 3B is a schematic illustration of a trash can filled with its contents, according to an embodiment of the present invention; and

FIG. 4 is an exemplary overall indication signal sent by the transceiver to the control center.

### DETAILED DESCRIPTION OF THE PREFERRED EMBODIMENTS

Hereinafter, when the term “trash can” is mentioned, it should be understood that it refers also to “trash bin” and vice-versa. Also, the terms “cellular phone” and “cell phone” are used interchangeably.

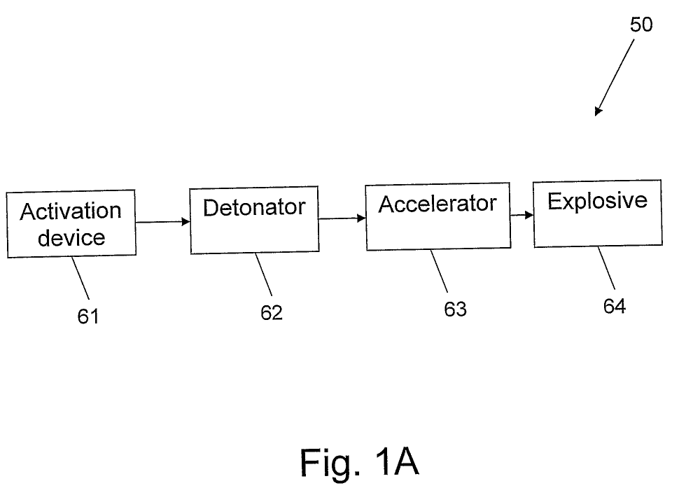

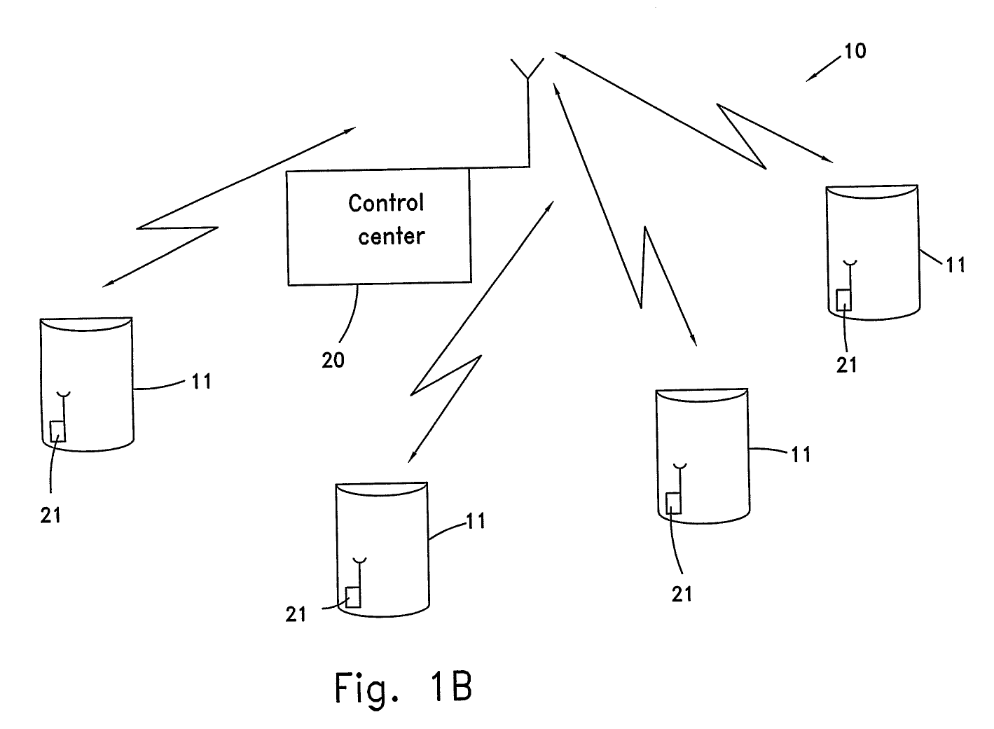

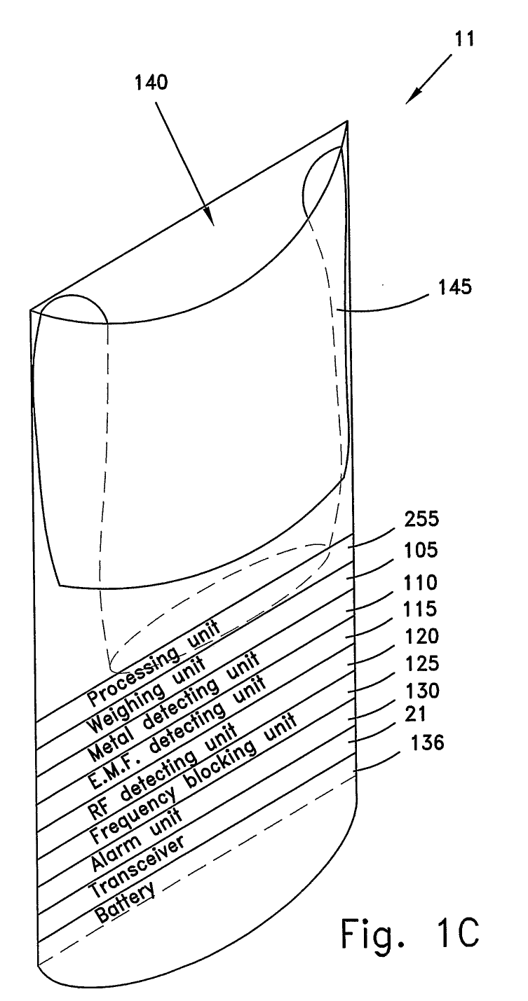

FIG. 1A is a schematic block diagram of a conventional bomb. Bomb 50 comprises activation device 61, detonator 62, accelerator 63 and explosive 64. Activation device 61 may be, for example, a cellular phone, a clock, or it may comprise chemical components for initiating a chemical reaction for activating said detonator. Detonator 62 can be based, for example, on acid or lead. Accelerator 63 is used for urging the ignition of explosive 64. Explosive 64 can be, for example, TATP (Triacetonetriperoxide), NG (Nitroglycerine), TNT (Trinitrotoluene) etc.

FIG. 1B is a schematic illustration of a plurality of trash cans communicating with a control center 20, according to the present invention. The control center is controlled by security personnel who are responsible for public safety. Trash can 11 is spread, for example, all over the airport terminal or another public place. In case of danger trash can 11 sends alert signals by means of transceiver 21 to control center 20. When an alerting message is received at the control center, the security personnel can begin evacuating civilians from proximity of the targeted trash can or take any other measures for dealing with the emergency.

It should be noted that every predetermined period of time, the control center initiates a test of the specific trash cans 11 in order to ensure their intactness and workability.

FIG. 1C is a schematic illustration of a trash can 100, according to an embodiment of the present invention. The trash can 11 comprises a processing unit 255, weighing unit 105, Metal detecting unit 110, E.M.F. (Electromagnetic Field) detecting unit 115, RF (Radio Frequency) detecting unit 120, Frequency blocking unit 125, Alarm unit 130, transceiver 21 and at least one Battery 136. Hereinafter weighing unit 105, Metal detecting unit 110, E.M.F. detecting unit 115 and RF detecting unit 120 are defined as sensing units.

Weighing unit 105 is responsible for determining a change in weight of the objects within the trash can, which is caused by objects thrown through opening 140. In cases when the weight change is within a predetermined weight range, for example from 250 grams to 100 kilograms, or in case when the weight change is above the predetermined value, for example above 500 grams, then weighing unit 105, by means of transceiver 21 sends an alert signal to the control center 20. According to an embodiment of the present invention, in cases when an alert signal is sent to the central center by means of transceiver 21, alarm unit 130 sounds an alarm in order to warn the people in proximity of the trash can about a possible danger. The weighing unit may be, for example model “1022” of Tedea-Huntleigh Ltd. Company located in United States of America (USA) and said weighing unit may have, for example dimensions of 13 centimeters in length, 2.54 centimeters in width and 2.2 centimeters in height. Transceiver 21 may be, for example, a remote terminal unit “Radio Piccolo XR” of Motorola Inc. company located in USA. Transceiver 21 may have, for example dimensions of 7.19 centimeters in length, 5.56 centimeters in width and 1.63 centimeters in height.

Metal detecting unit 110 detects the presence of metal components inside the trash can 11. When a new metal component is detected within the trash can, the metal detecting unit by means of transceiver 21 sends an alerting signal to the control center. Metal detecting unit 110 may be, for example “M-scope” model of Fisher Labs (Fisher Research Laboratory) located in California, USA. Metal detecting unit 110 may have, for example dimensions of 16.5 centimeters in length, 7.4 centimeters in width and 3.5 centimeters in height.

E.M.F. detecting unit 115 detects the presence of an electromagnetic field inside the trash can 100. The electromagnetic field can be caused, for example, by a battery or accumulator or by any electrical circuit, such as electronic clock or cellular phone. In cases when such fields are detected, the E.M.F. detecting unit by means of transceiver 21 sends an alerting signal to the control center. E.M.F. detecting unit 115 may be, for example “Digital Stat-Arc 2 IS model 282IS”, “Digital Stat-Arc 3 model 282A-1” or “ELFLX detector” of Less EMF Inc. company located in New-York, USA. E.M.F. detecting unit 115 may detect, for example an electromagnetic field from 0 to 20 Kilovolt at 1 inch or from 5 Hertz to 2 Kilohertz and said E.M.F. detecting unit may have, for example dimensions of 10.7 centimeters in length, 6.1 centimeters in width and 2.3 centimeters in height.

RF detecting unit 120, such as a cellular phone detector detects RF transmission within the trash can. In cases when such transmission is detected, RF detecting unit 120 by means of transceiver 21 sends an alert signal to the control center. RF detecting unit 120 may be, for example “Communication Radiation Detector Receiver Board” of “Innovative Wireless Technologies” company located in USA. RF detecting unit 120 may detect, for example signals from 24 Megahertz to 1880 Megahertz and it may have, for example dimensions of 6.35 centimeters in length, 6.22 centimeters in width and 1.8 centimeters in height.

Frequency blocking unit 125 blocks the RF transmission, such as a cellular phone RF transmission, to and from an object thrown inside the trash can 100. Since many bombs are activated remotely by means of a phone call to a cellular phone connected to said bomb, the presence of the above frequency blocking unit 125 is very important. The frequency blocking unit must be set before its usage to block a predetermined range of radio frequencies or to block at least one radio frequency. For example, the frequency blocking unit may be set to block all or selected frequencies in the range of 800 MHz to 1.3 GHz, which are typical for cellular phone RF transmission. Furthermore, it must be decided what kinds of alerting signals turn on frequency blocking unit 125. For example, the frequency blocking unit may be turned ON all the time or it may be turned ON only in cases when at least one indication for possible explosive is found within the trash can. According to an embodiment of the present invention, processing unit 255 activates the Frequency blocking unit 125 whenever necessary or said Frequency blocking unit may be ON continuously. For example, if RF detecting unit 120 detects RF transmission within the trash can, processing unit 255 activates Frequency blocking unit 125, and it simultaneously sends an alert signal to the control center 20 via transceiver 21. It also may activate audio and/or visual alarm by means of alarm unit 130. Frequency blocking unit 125 may be, for example a mobile phone blocker (cellular calls jammer) C-3000 of Cavadino & Co Limited company located in Hong Kong. Frequency blocking unit 125 may block, for example a signal from a cellular phone attached to bomb 50 (FIG. 1A), said signal being, for example from 800 Megahertz to 900 Megahertz. The frequency blocking unit may have, for example dimensions of 11.8 centimeters in length, 6 centimeters in width and 5.8 centimeters in height.

It should be noted, that Frequency blocking unit 125 may comprise a metal shield for preventing any RF communication to or from the trash can, excluding of course, communication of transceiver 21.

According to an embodiment of the present invention, battery 136 is any conventional battery for providing electric power to all the power-consuming units of the system. It should be noted that one or more units of the proposed system 100 can have their own rechargeable and/or replaceable batteries.

Also, it should be noted that all units of the proposed system must be adjusted or set to the desirable predetermined sensitivity values or to the predetermined ranges before their usage. For example, the weighing unit must be set to indicate emergency if the detected change in weight is within a predetermined range of 0.25 kilogram and above. Also, for example, the metal detecting unit may be set to indicate emergency only in cases when the detected metal components have relatively large dimensions and are not small like a pin or clip.

According to an embodiment of the present invention, the alarm, which is activated by means of alarm unit 130 also includes a visual alarm, such as revolving or flashing lights.

According to an embodiment of the present invention, the control center is provided with means for sending a signal for stopping the audio or visual alarm.

According to another embodiment of the present invention, the audio or visual alarm is stopped directly from the trash can by means of at least one switch.

According to still another embodiment of the present invention, the control center can remotely adjust and calibrate settings, parameters and sensitivity of the weighing unit, metal detecting unit, Electromagnetic Field detecting unit, RF detecting unit, Frequency blocking unit, alarm unit, processing unit and transceiver.

According to a further embodiment of the present invention, the settings, parameters and sensitivity of the weighing unit, metal detecting unit, Electromagnetic Field detecting unit, RF detecting unit, Frequency blocking unit, alarm unit, processing unit and transceiver are adjusted and calibrated, according to the needs, at the trash can.

According to still further embodiment of the present invention, after finding an indication of a suspicious article at the trash can, processing unit 225 may send an SMS (Short Message Service) message, for example, to beepers, Palms or PDAs (Personal Digital Assistants) of security personnel in order to warn them about the possible danger. However, in that case transceiver 21 has to include such a capability. It should be noted, that SMS messages also may be sent from the control center.

According to a still further embodiment of the present invention, all the units of the trash can 11 are in Sleep Mode, saving electrical power and they are activated only upon determination that a new article has been thrown into the trash can.

Preferably, processing unit 255 checks the state of battery 136 and sends a signal to the control center in case of low battery power.

It should be noted, that processing unit 255 is programmed to process all the data received from all the various units: weighing unit 105, Metal detecting unit 110, E.M.F. (Electromagnetic Field) detecting unit 115, RF (Radio Frequency) detecting unit 120, Frequency blocking unit 125, Alarm unit 130, transceiver 21 and at least one Battery 136. As previously said, that processing unit processes all the data received from all the various units, and determines whether there is a need to send an alarm signal to the control center, or not. For example, if only a metal object is found, without any further suspicious indication from the other units, there is probably no need to activate an alarm signal. However, if suspicious indications come from more than one unit, an alarm signal will obviously be sent, and probably also the audio and visual alerts will be activated.

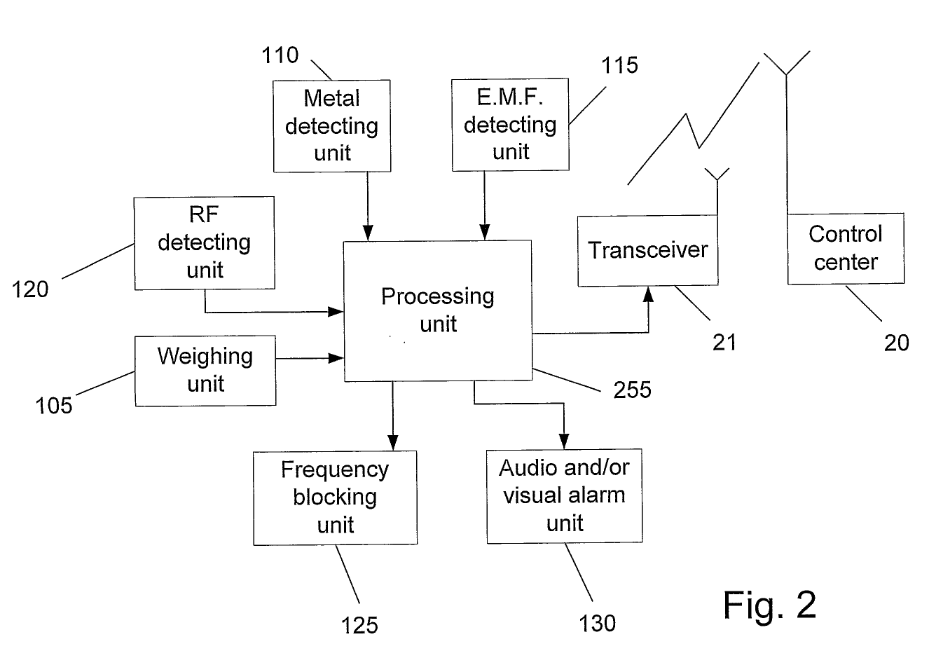

FIG. 2 is a schematic block diagram of trash can units, according to an embodiment of the present invention. The metal detecting unit 110, checks whether the thrown object comprises metal parts or components within the metal detecting unit sensitivity range. If the metal detecting unit detects the presence of metal parts, an indication is provided to the processing unit 255. The E.M.F. (Electromagnetic Field) detecting unit 115 checks for the presence of an electromagnetic field. The electromagnetic field can emanate from any device that consumes power from a battery. Such a device may be a clock, cellular phone, an electronic device communicating with a remote control, etc. It should be noted that any conventional activation device 61 (see FIG. 1) must use at least some kind of electronic device that consumes electricity, and therefore operation of such a device can be detected. If the E.F.M. detecting unit detects such a field, it sends an indication to processing unit 255. RF (Radio Frequency) detecting unit 120 checks for the presence of RF transmission from a cellular phone, for example. If RF detecting unit 120 detects RF transmission, it also sends an indication signal to processing unit 255. Weighing unit 105 determines whether there has been a change in the weight of the objects within the trash can 11, said change caused by objects thrown into said trash can, and sends indication about said change in weight to processing unit 255. If the weight change falls within a predetermined weight range, for example from 250 grams to 10 kilograms, or in cases when the weight change is above the predetermined value, for example above 500 grams, then processing unit 255 may send an alert signal to control center 20 by means of transceiver 21. The purpose of Frequency blocking unit 125 is to block the RF transmission to and from an object such as a cellular phone within the trash can. By blocking an incoming phone call to the cellular phone assumed to be connected to a bomb, frequency blocking unit 125 prevents bomb activation. As previously stated, processing unit 255 receives signals from metal detecting unit 110, E.M.F. detecting unit 115, RF detecting unit 120 and weighing unit 105. Processing unit 255 activates frequency blocking unit 125 in cases when RF transmission has been detected by means of RF detecting unit 120. Transceiver 21 transmits and receives signals to and from control center 20. Furthermore, processing unit 255 comprises a programmable memory, which can be programmed according to user's needs. For example, the processing unit 255 can be programmed to send an alert signal to control center 20 when the weighing unit detects the change in weight above 100 grams. Processing unit 255 receives data from all detecting units and from the weighing unit within the trash can and decides according to the data stored in its memory what to do next, whether to send alerting message to the control center, to activate the frequency blocking unit, to activate audio and/or visual alarm etc.

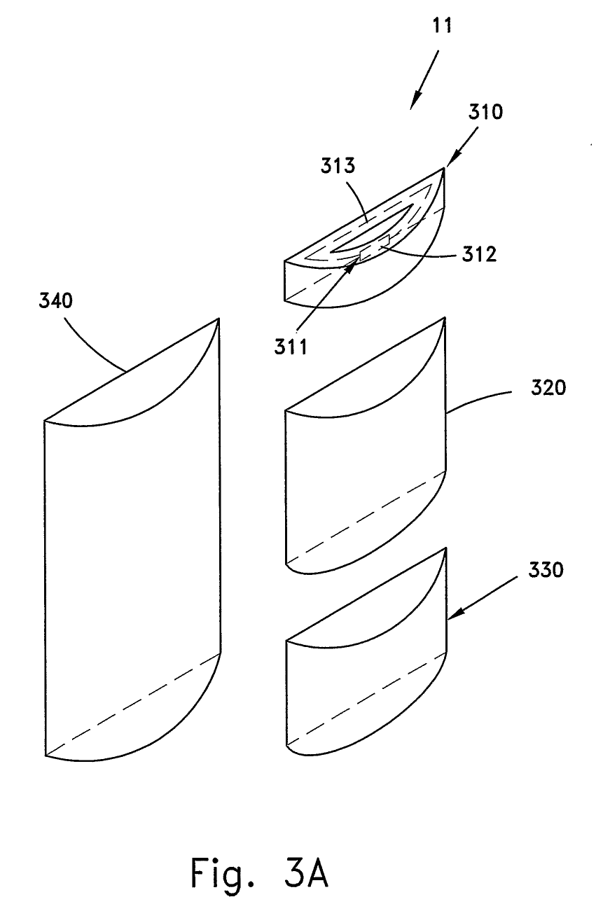

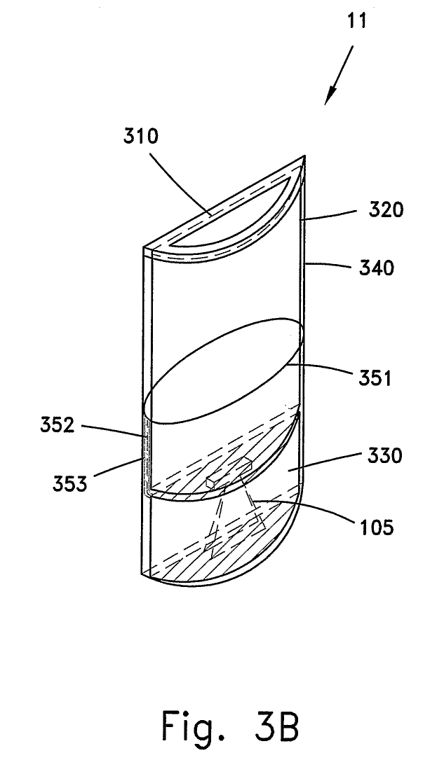

FIG. 3A is a schematic illustration of trash can contents, according to an embodiment of the present invention. Weighing unit 105, E.M.F. detecting unit 115, RF detecting unit 120, Frequency blocking unit 125, Alarm unit 130, processing unit 255, transceiver 21 and Battery 136 are located inside unit 330. Unit 330 is placed at the bottom of trash can body 340 and its content is preferably invisible to anyone passing near the trash can or looking inside the trash can. Unit 320 is used for gathering trash thrown into the trash can, and it is placed on the top of unit 330 inside trash can body 340. Since unit 320 is placed on the top of unit 330, weighing unit 105 located inside unit 330 determines a change in weight of the objects within unit 320, which is caused by objects thrown into said unit 320. Metal detecting unit 311 is located on the internal side of the trash can cover 310 and is invisible to anyone passing near the trash can, looking inside said trash can etc. Metal detecting unit 311 may comprise a body 312, processing data and alerting to control center in case of danger, and at least one wire surrounding trash can cover 310 for detecting metal objects thrown inside said trash can. According to an embodiment of the present invention, metal detecting unit 311 is connected to processing unit 255 (FIG. 2) and/or to battery 136 (FIG. 1C), located inside unit 330, by means of an additional at least one wire shielded by means of a shielding sleeve.

FIG. 3B is a schematic illustration of a trash can 11 filled with its contents, according to an embodiment of the present invention. For better illustration it is assumed that the front wall of the trash can is transparent. Unit 330 comprises weighing unit 105, E.M.F. detecting unit 115, RF detecting unit 120, Frequency blocking unit 125, Alarm unit 130, processing unit 255, transceiver 21 and Battery 136. Metal detecting unit 311 is located inside trash can cover 310. Weighing unit 105 measures a change in weight of the object thrown into unit 320. Unit 320 is placed over unit 330 and it does not have any friction with either trash can body 340 or with any other unit within trash can 11. Also, unit 330 does not have any friction with either trash can body 340 or with any other unit within trash can 11.

According to an embodiment of the present invention, E.M.F. detecting unit 115, located inside unit 330, comprises at least one wire 351, surrounding trash can body 340 for detecting the electromagnetic field. According to an embodiment of the present invention, E.M.F. detecting unit 115 detects an electromagnetic field within the trash can, but it is so designed not to sense E.M.F. caused by the legitimate units of the system. Units 310 and 330 comprising electronic units of the system may be shielded in order that E.F.M. detecting unit will be not able to detect E.M.F. caused by them. According to another embodiment of the present invention, E.M.F. detecting unit 115 also detects an electromagnetic field of the objects located some distance below and/or above wire 351, for example x centimeters below wire 351 and/or y centimeters above said wire.

The sensitivity of E.M.F. detecting unit may be set and adjusted according to user's needs. Additionally at least one wire 352 connects at least one wire 351 to E.M.F. detecting unit body 115 located inside unit 330. According to an embodiment of the present invention, wire 352 is shielded by means of a shielding sleeve 353.

In still another embodiment of the invention, the trash can also comprises a sensing unit for detecting a dirty bomb. Such sensing unit may comprise, for example, a Geiger counter for measuring and detecting radio activity, or other sensors for detecting a dirty bomb. Upon detection of a dirty bomb, an alert signal is initiated or transmitted as discussed above.

It should be noted, that all units of the trash can, including trash can body 340 and trash can cover 310, can have any form, such as circular, rectangular, triangular, polygonal, etc. Also, unit 330 can be a plate located on the top of weighing unit 105. According to another embodiment of the present invention, unit 320 does not present within the trash can.

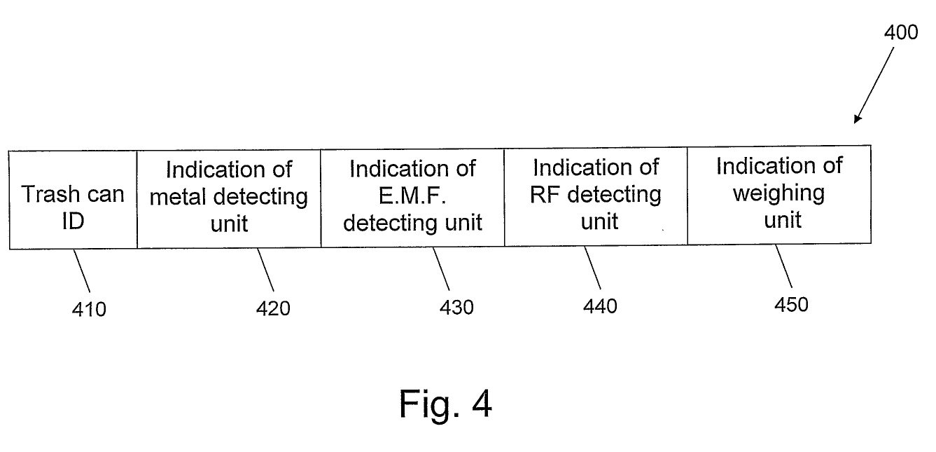

FIG. 4 is an exemplary overall indication signal 400 sent by transceiver 21 to control center 20 (FIG. 1B). The signal comprises an ID 410 (Identification number) of a specific trash can (FIG. 1B) sending the indication signal, and one or more indication signals received from the sensing unit: weighing unit 105, metal detecting unit 110, E.M.F. detecting unit 115 and RF detecting unit 120 (FIG. 1C). In cases, when the metal detecting unit detects the presence of metal components among the objects thrown into the trash can, overall indication signal 400 will comprise an indication 420 of said detection. Similarly, in overall indication signal 400 the field numbered 430 is indication of the E.M.F. detecting unit, the field numbered 440 is an indication of the RF detecting unit and the field numbered 450 is an indication of the weighing unit.

While some embodiments of the invention have been described by way of illustration, it will be apparent that the invention can be put into practice with many modifications, variations and adaptations, and with the use of numerous equivalents or alternative solutions that are within the scope of persons skilled in the art, without departing from the spirit of the invention or exceeding the scope of the claims.

1. A system for detecting bombs which are thrown into a trash can, comprising:
a. at least one sensing unit for sensing predetermined characteristics of objects thrown into the trash can, and forming an indication signal when a characteristic at a level predefined to be suspected is sensed; and
b. a processing unit for receiving indication signals from said at least one sensing unit, evaluating said signals, and whenever determining emergency, forming an alert signal;
characterized in that one of said sensing units is an RF detecting unit for detecting radio transmission from objects thrown into the trash can.

2. System according to claim 1, wherein the additional sensing units are selected from a group comprising:
a. a weighing unit;
b. a metal detecting unit;
c. an E.M.F. detecting unit; and
d. a sensing unit for detecting a dirty bomb.

3. System according to claim 1, further comprising a frequency blocking unit for blocking at least one radio frequency or for blocking a predetermined range of radio frequencies.

4. System according to claim 1, further comprising a transceiver for communicating with a control center, which is controlled by security personnel who are responsible for public safety.

5. System according to claim 1, further comprising an alarm unit for initiating an audio or visual alarm whenever determining emergency.

6. System according to claim 1, further comprising at least one battery or accumulator for providing electrical power to one or more units within said trash can.

7. System according to claim 1, wherein the trash can is further connected to a power supply providing electrical power to at least one unit within said trash can.

8. System according to claim 1, wherein the processing unit further sends one or more signals to one or more sensing units.

9. System according to claim 3, wherein the processing unit further sends one or more signals to the frequency blocking unit.

10. System according to claim 4, wherein the transceiver sends an alert signal to the control center when the change in weight, detected by means of the weighing unit is beyond a predetermined value or within a predetermined range.

11. System according to claim 4, wherein the transceiver sends an alert signal to the control center when one or more metal components in one or more objects thrown in the trash can are detected by means of the metal detecting unit.

12. System according to claim 4, wherein the transceiver sends an alert signal to the control center when the Electromagnetic Field inside the trash can is detected by means of the E.M.F. detecting unit.

13. System according to claim 4, wherein the transceiver sends an alert signal to the control center when the Radio Frequency transmission inside the trash can is detected by means of the RF detecting unit.

14. System according to claim 5, wherein the control center is further provided with means for stopping the audio or visual alarm.

15. System according to claim 5, wherein the audio or visual alarm is stopped at the trash can by means of at least one switch.

16. System according to claim 2, wherein the control center remotely adjusts and calibrates settings, parameters and sensitivity of the weighing unit, metal detecting unit, Electromagnetic Field detecting unit, RF detecting unit and Frequency blocking unit.

17. System according to claim 4, wherein the control center remotely adjusts and calibrates settings of the transceiver.

18. System according to claim 4, wherein the control center remotely adjusts and calibrates settings of the alarm unit.

19. System according to claim 2, wherein the settings, parameters and sensitivity of the weighing unit, metal detecting unit, Electromagnetic Field detecting unit, RF detecting unit and frequency blocking unit are adjusted and calibrated at the trash can.

20. System according to claim 4, wherein the settings, parameters and sensitivity of the transceiver are adjusted and calibrated at the trash can.

21. System according to claim 5, wherein the settings, parameters and sensitivity of the alarm unit are adjusted and calibrated at the trash can.

22. System according to claim 1, wherein the Frequency blocking unit comprises a metal shield for preventing any RF communication to or from the trash can, excluding communication of the transceiver.

23. System according to claim 2, wherein the weighing unit, metal detecting unit, Electromagnetic Field detecting unit, RF detecting unit and frequency blocking unit, are in Sleep Mode, saving electrical power, until at least one object is thrown into the trash can.

24. System according to claim 4, wherein the transceiver further comprises a programmable memory.

25. A method for detecting bombs which are thrown into a trash can, comprising:
a. sensing predetermined characteristics of objects thrown into the trash can by means of at least one sensing unit, and forming an indication signal by means of said at least one sensing unit when a characteristic at a level predefined to be suspected is sensed; and
b. receiving indication signals from said at least one sensing unit, evaluating said signals, and whenever determining emergency, forming an alert signal by means of a processing unit;
characterized in that one of said sensing units is an RF detecting unit for detecting radio transmission from objects thrown into the trash can.

26. Method according to claim 25, the additional sensing units are from a group comprising:
a. a weighing unit;
b. a metal detecting unit;
c. an E.M.F. detecting unit; and
d. a sensing unit for detecting a dirty bomb.

27. Method according to claim 25, further comprising providing a frequency blocking unit for blocking at least one radio frequency or for blocking a predetermined range of radio frequencies.

28. Method according to claim 25, further comprising providing a transceiver for communicating with a control center, which is controlled by security personnel who are responsible for public safety.

29. Method according to claim 25, further comprising providing an alarm unit for initiating an audio or visual alarm whenever determining emergency.

30. Method according to claim 25, further comprising providing at least one battery or accumulator for providing electrical power to one or more units within said trash can.

31. Method according to claim 25, further comprising connecting the trash can to a power supply, providing electrical power to at least one unit within said trash can.

32. Method according to claim 25, further comprising sending one or more signals to one or more sensing units by means of the processing unit.

33. Method according to claim 27, further comprising sending one or more signals to the frequency blocking unit by means of the processing unit.

34. Method according to claim 26, further comprising sending an alert signal to the control center by means of a transceiver, when the change in weight, detected by means of the weighing unit is beyond a predetermined value or within a predetermined range.

35. Method according to claim 26, further comprising sending an alert signal to the control center by means of a transceiver, when one or more metal components in one or more objects thrown in the trash can are detected by means of the metal detecting unit.

36. Method according to claim 26, further comprising sending an alert signal to the control center by means of a transceiver, when the Electromagnetic Field inside the trash can is detected by means of the E.M.F. detecting unit.

37. Method according to claim 26, further comprising sending an alert signal to the control center by means of a transceiver, when the Radio Frequency transmission inside the trash can is detected by means of the RF detecting unit.

38. Method according to claim 29, further comprising providing means for stopping the audio or visual alarm within the control center.

39. Method according to claim 29, further comprising stopping the audio or visual alarm at the trash can by means of at least one switch.
